(19) United States
(12) Patent Application Publication
(10) Pub. No.: US 2024/0165355 A1
(43) Pub. Date: May 23, 2024

(54) ENDOTRACHEAL TUBE HAVING PARTIAL WIRE REINFORCEMENT

(71) Applicant: Seattle Children’s Hospital d/b/a Seattle Children’s Research Institute, Seattle, WA (US)

(72) Inventor: Agnes Hunyady, Seattle, WA (US)

(21) Appl. No.: 18/517,710

(22) Filed: Nov. 22, 2023

Related U.S. Application Data
(60) Provisional application No. 63/384,859, filed on Nov. 23, 2022.

(51) Int. Cl. A61M 16/04

(52) U.S. Cl. CPC A61M 16/0477

(57) ABSTRACT

An endotracheal tube having a bendable portion is provided. The endotracheal tube can include a first tube portion at a distal end of the endotracheal tube, a bendable second tube portion at a proximal end, a transition region located therebetween, and a reinforcing component positioned along the bendable second tube portion. The endotracheal tube can have a central airflow passageway extending from the proximal end to the distal end, with the reinforcing component configured to maintain a minimum airflow area of the central airflow passageway within the second tube portion when the second tube portion is in a bent configuration. The tube can include an inner wall and an outer wall to surround the reinforcing component. The bent configuration of the tube can include a bend having an angle from about 20° to 160°.

### CROSS-REFERENCE TO RELATED APPLICATION

This application claims the benefit of Provisional Application No. 63/384,859, filed Nov. 23, 2022, the entire disclosure of which is hereby incorporated by reference herein for all purposes.

### BACKGROUND

Many oral and maxillofacial surgeries require placing an endotracheal tube in the midline of the patient in such a way that the tube exits the trachea through the voice box, following the sagittal contour of the pharynx then the oral cavity, but then exiting the mouth and bending the tube away from the surgical field. During use, the endotracheal tube can require bending of 90° or greater over the gums, teeth, or back of the nose of the patient. These relatively sharp bends can cause kinking of the tube, restricting airflow through the tube and depriving the patient of necessary oxygen delivery. Some endotracheal tubes are designed to prevent such kinking by including wire reinforcement into the wall of the tube (e.g., RÜSCH® Endotracheal Tubes), or by preforming a bend in the tube during manufacturing at a specified length from the proximal end (e.g., SHILEY® Oral and Nasal RAE Endotracheal Tubes).

Current technology reinforced tubes have increased wall thickness along the length of the tube to accommodate wire reinforcement. In a patient, airway resistance is inversely proportional to the fourth power of the radius of the airway. Accordingly, a relatively small decrease in the lumen (central airflow passageway) of an endotracheal tube due to increased wall thickness can significantly increase airway resistance. In practice, the outer diameter of a reinforced tube can be too large for a pediatric trachea while keeping the lumen large enough for proper airflow. It is therefore important to minimize the wall thickness of the endotracheal tube, particularly in endotracheal tubes designed for children where the small diameter of the trachea, the glottis, and the subglottis limits the maximum outer diameter of the endotracheal tube.

Preformed tubes have a bend at a fixed distance from the distal end of the tube. During pediatric body development, the cross section of the airway does not necessarily grow at the same rate as the length of the airway. Instead, the diameter of the airway correlates with age, while the airway length correlates more closely with height. Further, airway pathologies can separately affect tracheal diameter and tracheal length. As such, the bend location of preformed tubes can cause suboptimal positioning of the proximal (patient) end of the tube, compromising patient safety.

### DESCRIPTION OF THE DRAWINGS

The disclosure will be better understood, and other details, characteristics and advantages of the disclosure will appear more clearly when reading the following description made as a non-limiting example and with reference to the annexed drawings in which:

FIG. 1 is a perspective view of an endotracheal tube in accordance with embodiments of the present disclosure;

FIG. 2 is an exploded perspective view of the endotracheal tube of FIG. 1;

FIG. 3 is a side view of the endotracheal tube of FIG. 1, shown in a neutral configuration;

FIG. 4 is a side view of the endotracheal tube of FIG. 1, shown in a bent configuration;

FIG. 5 is a front view of the endotracheal tube of FIG. 1; and

FIG. 6 is a cross-sectional side view of the endotracheal tube of FIG. 1.

### DETAILED DESCRIPTION

The detailed description set forth below in connection with the appended drawings, where like numerals reference like elements, is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the claimed subject matter to the precise forms disclosed.

Embodiments of the present disclosure include endotracheal tubes having partial wire reinforcement in portions of the tube where relatively tight bend radii are required, e.g., at a proximal end of the tube located at the mouth of the patient when the tube is in position. In this regard, the proximal end of the endotracheal tube is reinforced to permit bending without kinking that would restrict airflow through the tube. The partial wire reinforcement of tubes of the present disclosure can allow constant inner diameter in the lumen (central airflow passageway) along substantially the entire length of the tube to minimize airflow resistance during use. Lower airflow resistance permits delivering lower air pressure through the endotracheal tube, which can reduce high pressure-related lung or bronchial injury. While embodiments of the present disclosure can increase airway stability and surgical access during maxillofacial/otorhinolaryngological/dental procedures, other applications may include airway support for pediatric patients with congenital abnormalities.

In another aspect, present disclosure includes methods of determining patient size ranges for the transition region of the reinforced portion to unreinforced tubing along the length of the endotracheal tube.

Among others, embodiments of the endotracheal tube are expected to have the following benefits: (1) permitting smaller diameters on the distal, unreinforced end entering the trachea to better fit pediatric patients; 2) providing tube flexibility in the oral region of the patient without kinking that restricts airflow through the lumen; 3) minimizing air resistance through the lumen; 4) providing patient size related reinforcement transition region and overall lengths for improved patient fitment; (5) minimizing part variation to fit patient anatomy variation.

Embodiments of the present disclosure can include a smooth internal and external surfaces at the transition region between reinforced and unreinforced tubing of the endotracheal tube. The present disclosure provides endotracheal tubes with wire reinforcement at a portion of the tube positioned at the proximal end, permitting smaller bending radii in the reinforced portion while minimizing kinking. The length of the reinforced portion with respect to the overall length of the endotracheal tube can be tailored to fit various anatomical sizes within the pediatric patients. While the outer diameter of the endotracheal tube is necessarily larger in the reinforced portion (oral), the inner diameter of the lumen is substantially the same as the inner diameter in the unreinforced portion (transtracheal). For oral endotracheal tube configurations, the reinforced proximal portion is intended to be orally located within the pediatric patient in the mouth and/or near the back of the nose in which the anatomy of the patient does not restrict the outer diameter as severely as within the trachea. For nasal endotracheal tube configurations, the reinforced proximal portion is intended to be nasally located.

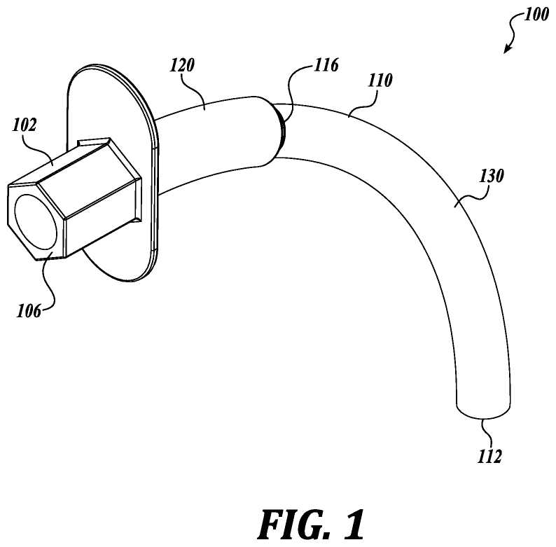

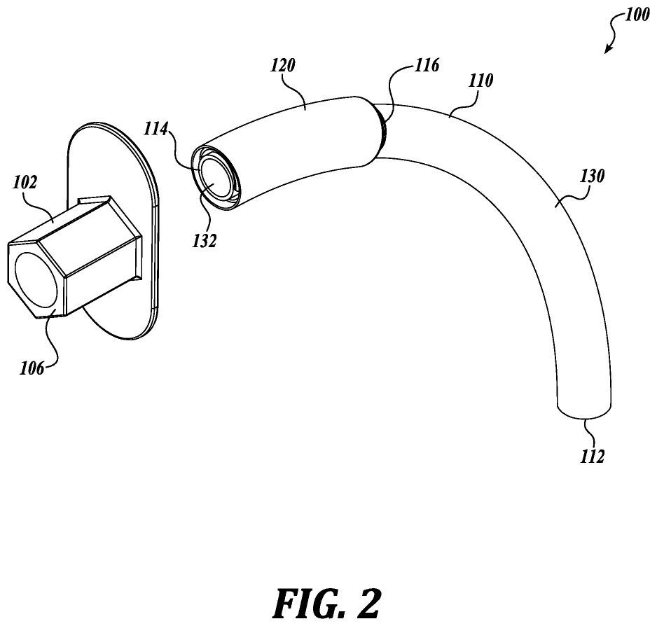

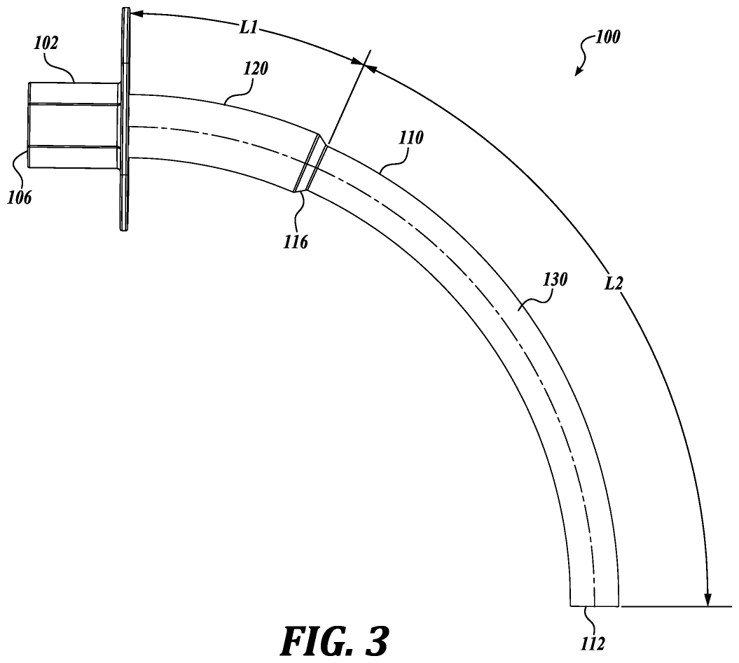

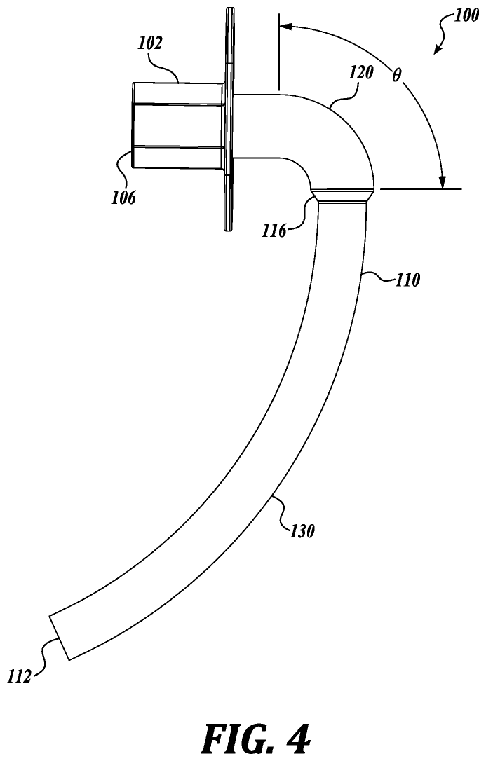

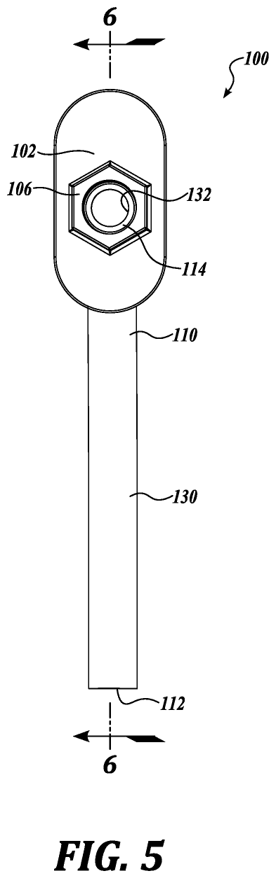

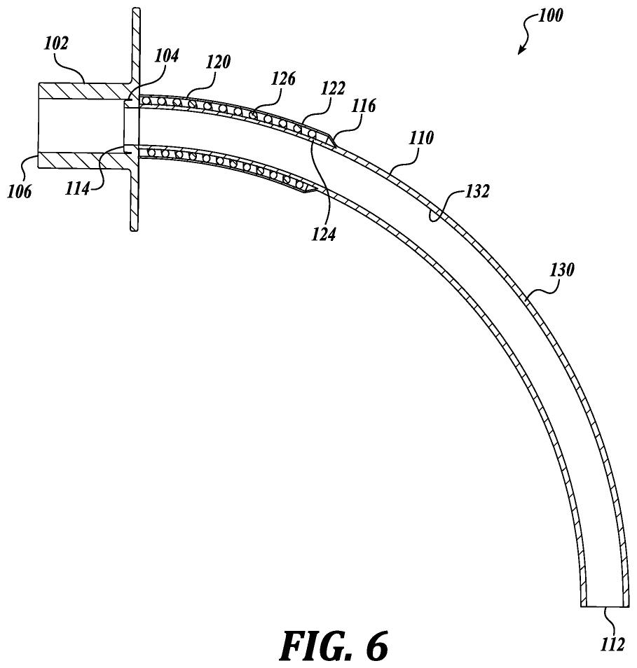

FIGS. 1 and 2 are assembled and exploded perspective views, respectively, of an endotracheal tube assembly 100 (“assembly 100”), FIGS. 3 and 4 are side views and FIG. 5 is a front view of the assembly 100, and FIG. 6 is a cross-sectional view of the assembly 100, each configured in accordance with embodiments of the present disclosure. The assembly 100 can include a connector/adaptor portion 102 (“connector 102”) and an endotracheal tube 110 (“tube 110”) removably coupled to an interface portion 104 (see FIG. 6) of the connector 102. The connector 102 can be configured to fluidly couple the tube 110 to a medical component providing airflow for the patient, e.g., tubing of a mechanical ventilator, a manual resuscitator or self-inflating bag (AMBU® bag, etc.). In some embodiments, the connector can include a universal port 106 (e.g., a 15 mm port configured to receive a 15 mm mechanical ventilator tube). In other embodiments, the connector 102 can include other port and attachment configurations, e.g., an anesthesia port for connection to an anesthesia circuit (not shown). The connector 102 can couple to the tube 110 at the interface portion 104 using any suitable mechanical connection, e.g., threaded, press-fit, twist-to-lock, adhesive, etc., or can be molded together during manufacturing. Embodiments of the assembly 100 can be configured such that various connectors 102 are usable with each tube 110, which can improve compatibility of the assembly 100 with various airflow sources. Although one example of a connector is shown in the FIGURES, any suitable connector can be used with the endotracheal tubes of the present disclosure.

The tube 110 includes a distal end 112 and a proximal end 114, with the proximal end 114 interfacing the connector 102. When inserted into the patient, the distal end 112 is intended to be positioned near a carina of the patient (the bifurcation of trachea). The tube 110 can include a bendable, reinforced portion 120 near the proximal end 114 and an unreinforced portion 130 extending from the reinforced portion 120 to the distal end 112, and having a transition region 116 therebetween. In the embodiments of the present disclosure, the reinforced portion 120 is intended to be positioned in the oral region of the patient, and the unreinforced portion is intended to be positioned in the tracheal region of the patient. As shown in FIG. 2, the tube 110 can include a central airflow passageway 132 (“lumen 132”) extending through the reinforced and unreinforced portions 120 and 130.

The reinforced portion 120 and unreinforced portion 130 of the tube 110 can generally be curved (arcuate) in a neutral configuration (e.g., the configuration shown in FIGS. 1-3, 5, and 6) to aid insertion into the patient. Although the FIGURES show one possible curve configuration, embodiments of the present disclosure are suitable for use with any curvature configuration, including nonconstant curvatures, different curvatures in different portions of the tube 110 (e.g., a curved unreinforced portion 130 and a straight reinforced portion 120, or vice versa), and/or with a straight configuration through the tube 110. Referring to FIG. 3, the tube 110 can have an arclength L1 corresponding to the curved length of the reinforced portion 120 and an arclength L2 corresponding to the curved length of the reinforced portion 130. In tubes having a straight configuration, the lengths L1 and L2 are linear. The transition region 116 is located a length of L1 away from the proximal end 114, and the transition region 116 is located a length of L2 from the distal end 112. The L1 and L2 lengths can be varied to fit different anatomical sizes within the patient.

FIG. 4 shows a bent configuration of the tube 110 with a bend in the bendable, reinforced portion 120. The bent configuration of FIG. 4 is intended to show one representative example of the assembly 100 in use within the patient with the reinforced portion 120 bent away from the surgical field. The bend can be defined with an angle θ, but can have any suitable bend radius or bend angle, which will be dictated by the various specifications of the reinforced portion 120 (material, diameter, wall thickness, length, reinforcing component (described below), etc.). In some embodiments, the bend angle θ is from about 20° to 160°, about 70° to 160°, about 90° to 160°, or about 90°. Bending of the reinforced portion 120 during use within a patient may cause increased curvature to other areas of the tube 110, and likewise, insertion of the tube 110 into the patient may change the curvature of the tube 110 upon interaction with the anatomy of the patient.

Turning to FIG. 6, the reinforced portion 120 can include an outer wall 122, and inner wall 124, and a reinforcing component 126 positioned therebetween. In the illustrated embodiment, the reinforcing component 126 is a coil spring extending from the transition region 116 toward the proximal end 114. In other embodiments, the reinforcing component 126 can be any suitable configuration to reduce kinking (e.g., braided hose section, a sleeve, circumferential rings, longitudinal or coaxial wires, etc.) in the tube 110 when bending the reinforced portion 120 away from the surgical field, such as in the bent configuration shown in FIG. 4. Likewise, the reinforcing component 126 can be any suitable material to reduce kinking while considering, e.g., strength to weight ratio, material cost, availability, toxicity, flexibility, among other considerations. Suitable materials can include metal, plastics (e.g., deformable), composite (e.g., carbon fiber, Kevlar), among others. The outer wall 122 and the inner wall 124 can converge at the transition region 116, where the tube 110 transitions to the unreinforced portion 130. In some embodiments, either the outer wall 122 or the inner wall 124 can be omitted. In the illustrated embodiment, the lumen 132 has a constant diameter along the length of the tube 110 from the distal end 112 to the proximal end 114; however, in other embodiments, the diameter the lumen 132 may change along the length the tube 110, e.g., at the transition region 116.

As will be described below, the length of the reinforced portion 120 with respect to the overall length of the tube 110 can be tailored to fit various anatomical sizes within the pediatric patients. In this regard, the reinforced portion 120 is intended to be positioned in the oral region of the patient without entering the tracheal region, wherein the outer diameter is more restricted, particularly in pediatric patient anatomies. Similarly, the unreinforced portion 130 is intended to be positioned in the tracheal region of the patient without impacting the carina area or entering the right or left mainstem bronchi.

Various diameters and lengths of the tube 110 and locations of the transition region 116 can be provided to fit a wide variety of patient anatomies. Embodiments of the assembly 100 can be configured to fit ranges in distance from the carina to the gums and nares of substantially all age, weight, and height groups in pediatric patients. This distance, denoted herein as “Front Teeth to Carina” or “FTC,” provides guidance for the location of the transition region 116 along the length of the tube 110. For example, a patient being intubated with the tube 110 would benefit from minimal airway resistance, reduced kinking or collapse in the bend region (reinforced portion 120), and proper length such that the tube 110 accommodates midline placement and a bend in the reinforced portion 120 at the gums or teeth. In nasal endotracheal tubes, the distance from the carina to the nare (nostril) of the nose provides guidance for the location of the transition region 116 along the length of the tube 110. Tables 1 and 2, below, have data for oral endotracheal tubes and nasal endotracheal tubes, respectively.

TABLE 1
Oral endotracheal tube data

| Tube | | | Medi- | | MinFTC- | | Medi- | | | Medi- | |
|---|---|---|---|---|---|---|---|---|---|---|---|
| Diameter | TotalCount | MinFTC | anFTC | MaxFTC | 1.5 cm | MinWeight | anWeight | MaxWeight | MinHeight | anHeight | MaxHeight |
| 3 | 36 | 9.8 | 11.15 | 12.6 | 8 | 1.9 | 3.8 | 5.9 | 45 | 53 | 63 |
| 3.5 | 25 | 10 | 13.1 | 17.4 | 9 | 2.1 | 6.6 | 13.8 | 45 | 63 | 92 |
| 4 | 17 | 13.3 | 14.8 | 16.4 | 12 | 7 | 11 | 15.6 | 64 | 78 | 103 |
| 4.5 | 10 | 15.9 | 17 | 18.2 | 14 | 13.5 | 17 | 22 | 92 | 105 | 118 |
| 5 | 12 | 17.1 | 18.55 | 20.1 | 16 | 15.6 | 19.65 | 25 | 106 | 116.5 | 125 |
| 5.5 | 11 | 18.5 | 20.7 | 21.7 | 17 | 21.4 | 28 | 37 | 123 | 128 | 142 |
| 6 | 28 | 20 | 22.95 | 25.4 | 19 | 26.6 | 42.5 | 76.5 | 134 | 153.5 | 172 |
| 6.5 | 11 | 22.7 | 24.5 | 27.2 | 21 | 47.2 | 57 | 70 | 155 | 167 | 175 |
| 7 | 7 | 23.6 | 25.8 | 26.9 | 22 | 52.4 | 59.3 | 71 | 162 | 168 | 174 |
| 8 | 1 | 29.2 | 29.2 | 29.2 | 24 | 180 | 180 | 180 | 185 | 185 | 185 |

TABLE 2
Nasal endotracheal tube data

| ETT.size | TotalCount | MinNoseCarina | MedianNoseCarina | MaxNoseCarina | MinNoseCarina-1 cm |
|---|---|---|---|---|---|
| 3 | 26 | 10.66693878 | 12.86 | 14.65 | 10 |
| 3.5 | 47 | 12.99 | 14.9 | 17.65 | 12 |
| 4 | 17 | 12.75932203 | 16.04 | 19.88 | 12 |
| 4.5 | 6 | 18.41 | 19.2 | 20.42915254 | 17 |
| 5 | 6 | 18.28 | 21.875 | 23.12861972 | 17 |
| 5.5 | 8 | 19.43 | 22.365 | 25.02 | 18 |
| 6 | 2 | 23.28 | 23.82 | 24.36 | 22 |
| 6.5 | 5 | 24.91 | 26.52 | 29.25 | 23 |
| 7 | 23 | 24.49 | 29.64 | 32.83 | 23 |

| ETT.size | MinWeight | MedianWeig [symbol] | MaxWeight | MinHeight | MedianHeight | MaxHeight |
|---|---|---|---|---|---|---|
| 3 | 2 | 3.3 | 5.69 | | | |
| 3.5 | 3.2 | 5.7 | 8.3 | 52 | 62 | 71 |
| 4 | 5.8 | 8.2 | 16.4 | 61 | 72.2 | 96.5 |
| 4.5 | 13.9 | 15.55 | 17.6 | 91.5 | 95.35 | 102.5 |
| 5 | 16.8 | 20.4 | 30.8 | 101 | 109.4 | 125 |
| 5.5 | 21.2 | 23.85 | 37.5 | 109 | 125 | 137.9 |
| 6 | 35.6 | 49.85 | 64.1 | 128.2 | 135.7 | 143.2 |
| 6.5 | 32.4 | 40.1 | 60.9 | 139.5 | 152.5 | 162.5 |
| 7 | 40.2 | 58.9 | 88.5 | 152.2 | 166 | 185 |

[symbol]

indicates data missing or illegible when filed

As shown in Table 1, above, oral endotracheal tube data is provided. In Table 1, the first column is the tube internal diameter in mm; the second column is the total count of the patients measured; the third column is the minimum FTC distance in the patients measured; the fourth column is the median FTC distance and the patients measured; the fifth column is the maximum FTC distance and the patients measured; the sixth column is the minimum FTC in cm, subtracting 1.5 cm to account for a gap between the tip of the endotracheal tube and the carina of the patient to avoid impacting the carina and/or accidental slipping of the tip of the tube into one of the main bronchi with movement of the head of the patient; the seventh column is the minimum weight of the patients measured; the eighth column is the median weight of the patients measured; the ninth column is the maximum weight of the patients measured; the tenth column is the minimum height of the patients measured; the eleventh column is the median height of the patients measured; the twelfth column is the maximum height of the patients measured.

As shown in Table 2, above, Nasal endotracheal tube data is provided. In Table 2, the first column is the tube (endotracheal tube is denoted “ETT”) diameter in cm; the second column is the total count of the patients measured; the third column is the minimum nose to carina distance in the patients measured; the fourth column is the median nose to carina distance and the patients measured; the fifth column is the maximum nose to carina distance and the patients measured; the sixth column is the minimum nose to carinaf in cm, subtracting 1.5 cm to account for a gap between the tip of the endotracheal tube and the carina of the patient to avoid impacting the carina and/or accidental slipping of the tip of the tube into one of the main bronchi with movement of the head of the patient; the seventh column is the minimum weight of the patients measured; the eighth column is the median weight of the patients measured; the ninth column is the maximum weight of the patients measured; the tenth column is the minimum height of the patients measured; the eleventh column is the median height of the patients measured; the twelfth column is the maximum height of the patients measured.

Embodiments of the present disclosure can be commercialized through manufacture and sales of a new product line of endotracheal tubes whose partial reinforcement section length varies with tube diameter as provided herein. Such tubes are expected to improve surgical access while maintaining proper airway support during oral surgeries. The variety of tube models currently available only partially address this need, which validates the commercial opportunity.

The present application may reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 10% of the stated value. For the purposes of the present disclosure, the phrase “at least one of A and B” is equivalent to “A and/or B” or vice versa, namely “A” alone, “B” alone or “A and B.”. Similarly, the phrase “at least one of A, B, and C,” for example, means (A), (B), (C), (A and B), (A and C), (B and C), or (A, B, and C), including all further possible permutations when greater than three elements are listed.

The principles, representative embodiments, and modes of operation of the present disclosure have been described in the foregoing description. However, aspects of the present disclosure which are intended to be protected are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure, as claimed.

1. An endotracheal tube, comprising:
a transition region located at an intermediate position along the endotracheal tube;
a first tube portion extending from the transition region to a distal end of the endotracheal tube;
a second tube portion extending from the transition region to a proximal end of the endotracheal tube;
a reinforcing component positioned along the second tube portion; and
a central airflow passageway extending from the proximal end to the distal end,
wherein the reinforcing component is configured to maintain a minimum airflow area of the central airflow passageway within the second tube portion when the second tube portion is in a bent configuration.

2. The endotracheal tube of claim 1, wherein the second tube portion further comprises an inner wall surrounding the central airflow passageway, and wherein the reinforcing component is positioned radially outward from the inner wall.

3. The endotracheal tube of claim 2, wherein the second tube portion further comprises an outer wall positioned radially outward from and surrounding the reinforcing component.

4. The endotracheal tube of claim 1, wherein the reinforcing component is selected from the group consisting of a coil spring, a braided hose section, a sleeve, circumferential rings, longitudinal wires, and coaxial wires.

5. The endotracheal tube of claim 1, wherein the central airflow passageway has a constant diameter from the proximal end to the distal end when the endotracheal tube is in a neutral configuration.

6. The endotracheal tube of claim 1, wherein the bent configuration includes a bend in the second tube portion having an angle from about 20° to 160°.

7. The endotracheal tube of claim 1, wherein the bent configuration includes a bend in the second tube portion having an angle from about 70° to 160°.

8. The endotracheal tube of claim 1, wherein the bent configuration includes a bend in the second tube portion having an angle from about 90° to 160°.

9. The endotracheal tube of claim 1, wherein the proximal end is configured to operably couple to a connector to fluidly couple the central airflow passageway to a medical component.

10. The endotracheal tube of claim 9, wherein the medical component is a mechanical ventilator, a manual resuscitator, or a self-inflating bag.

11. The endotracheal tube of claim 9, wherein the connector includes a universal port configured to receive a 15 mm ventilator tube.

12. An endotracheal tube, comprising:
a tube having a proximal end, a distal end, and a central airflow passageway open at the proximal and distal ends;
a reinforcing component positioned along a bendable portion of the tube at the proximal end; and
an outer wall positioned radially outward from and surrounding the reinforcing component,
wherein the reinforcing component is configured to maintain a minimum airflow area of the central airflow passageway when the bendable portion of the tube is in a bent configuration.

13. The endotracheal tube of claim 12, further comprising a transition region located at an end of the reinforcing component toward the distal end of the tube.

14. The endotracheal tube of claim 12, wherein the tube has a thinner wall thickness surrounding the central airflow passageway along the reinforcing component than along the remainder of the tube.

15. The endotracheal tube of claim 12, wherein the reinforcing component is selected from the group consisting of a coil spring, a braided hose section, a sleeve, circumferential rings, longitudinal wires, and coaxial wires.

16. The endotracheal tube of claim 12, wherein the central airflow passageway has a constant diameter from the proximal end to the distal end when the endotracheal tube is in a neutral configuration.

17. The endotracheal tube of claim 12, wherein the bent configuration includes a bend in the bendable portion having an angle from about 20° to 160°.

18. The endotracheal tube of claim 12, wherein the bent configuration includes a bend in the bendable portion having an angle from about 70° to 160°.

19. The endotracheal tube of claim 12, wherein the bent configuration includes a bend in the bendable portion having an angle from about 90° to 160°.

20. The endotracheal tube of claim 12, wherein the proximal end is configured to operably couple to a connector having a universal port to fluidly couple the central airflow passageway to a medical component.
